(19) United States
(12) Patent Application Publication
(10) Pub. No.: US 2008/0009786 A1
(43) Pub. Date: Jan. 10, 2008

(54) Drug-eluting Device for Treatment of Chronic Total Occlusions

(75) Inventor: Katherine G. Coughlin, Ipswich, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/859,302

(22) Filed: Sep. 21, 2007

Related U.S. Application Data
(62) Division of application No. 11/275,956, filed on Feb. 7, 2006.

(51) Int. Cl. A61M 31/00

(52) U.S. Cl. 604/60

(57) ABSTRACT

A drug-eluting medical device and method for treating a chronic total occlusion. The drug-eluting medical device is implanted into the chronic total occlusion and elutes a drug that softens or dissolves the plaque of the occlusion over a period of time. After the medical device has resided in the occlusion for an appropriate period of time such that at least a portion of the chronic total occlusion has been softened or dissolved, a guidewire can cross the occlusion and a procedure such as PTCA can be performed.

### FIELD OF THE INVENTION

The invention relates generally to intra-luminal devices for the treatment of chronic total occlusions (CTO) in a lumen, and more particularly, to a drug-eluting device and method for the treatment of CTO.

### BACKGROUND OF THE INVENTION

Stenotic lesions may comprise a hard, calcified substance and/or a softer thrombus material, each of which forms on the lumen walls of a blood vessel and restricts blood flow there through. Intra-luminal treatments such as balloon angioplasty (PTA, PTCA, etc.), stent deployment, atherectomy, and thrombectomy are well known and have proven effective in the treatment of such stenotic lesions. These treatments often involve the insertion of a therapy catheter into a patient's vasculature, which may be tortuous and may have numerous stenoses of varying degrees throughout its length. In order to place the distal end of a catheter at the treatment site, a guidewire is typically introduced and tracked from an incision, through the vasculature, and across the lesion. Then, a catheter (e.g. a balloon catheter), perhaps containing a stent at its distal end, can be tracked over the guidewire to the treatment site. Ordinarily, the distal end of the guidewire is quite flexible so that it can be rotatably steered and pushed through the bifurcations and turns of the typically irregular passageway without damaging the vessel walls.

In some instances, the extent of occlusion of the lumen is so severe that the lumen is completely or nearly completely obstructed, which may be described as a total occlusion. If this occlusion persists for a long period of time, the lesion is referred to as a chronic total occlusion or CTO. Furthermore, in the case of diseased blood vessels, the lining of the vessels may be characterized by the prevalence of atheromatous plaque, which may form total occlusions. The extensive plaque formation of a chronic total occlusion typically has a fibrous cap surrounding softer plaque material. This fibrous cap may present a surface that is difficult to penetrate with a conventional guidewire, and the typically flexible distal tip of the guidewire may be unable to cross the lesion.

Thus, for treatment of total occlusions, stiffer guidewires have been employed to recanalize through the total occlusion. However, due to the fibrous cap of the total occlusion, a stiffer guidewire still may not be able to cross the occlusion. Further, when using a stiffer guidewire, great care must be taken to avoid perforation of the vessel wall.

Further, in a CTO, there may be a distortion of the regular vascular architecture such that there may be multiple small non-functional channels throughout the occlusion rather than one central lumen for recanalization. Thus, the conventional approach of looking for the single channel in the center of the occlusion may account for many of the failures. Furthermore, these spontaneously recanalized channels may be responsible for failures due to their dead-end pathways and misdirecting of the guidewires. Once a “false” tract is created by a guidewire, subsequent attempts with different guidewires may continue to follow the same incorrect path, and it is very difficult to steer subsequent guidewires away from the false tract.

Another equally important failure mode, even after a guidewire successfully crosses a chronic total occlusion, is the inability to advance a balloon or other angioplasty equipment over the guidewire due to the fibrocalcific composition of the chronic total occlusion, mainly both at the “entry” point and at the “exit” segment of the chronic total occlusion. Even with balloon inflations throughout the occlusion, many times there is no antegrade flow of contrast injected, possibly due to the recoil or insufficient channel creation throughout the occlusion.

Atherosclerotic plaques vary considerably in their composition from site to site, but certain features are common to all of them. They contain many cells; mostly these are derived from cells of the wall that have divided wildly and have grown into the surface layer of the blood vessel, creating a mass lesion. Plaques also contain cholesterol and cholesterol esters, commonly referred to as fat. This lies freely in the space between the cells and in the cells themselves. A large amount of collagen is present in the plaques, particularly advanced plaques of the type which cause clinical problems. Additionally, human plaques contain calcium to varying degrees, hemorrhagic material including clot and grumous material composed of dead cells, fat and other debris. Relatively large amounts of water are also present, as is typical of all tissue.

Thus, there is a need for a method of treatment of the plaque of a CTO to facilitate guidewire passage through the occlusion as a prerequisite for successful angioplasty.

### BRIEF SUMMARY OF THE INVENTION

The present invention is a drug-eluting medical device that is inserted into a chronic total occlusion. After insertion, the medical device elutes a drug that softens or dissolves at least a portion of the plaque of the occlusion. After the medical device has resided in the occlusion for an appropriate period of time, a guidewire can cross the occlusion and a procedure such as PTCA can be performed.

The medical device of the present invention can be made of a material that is bioerodable, such that it dissolves in the vasculature as it releases the drug for softening or dissolving the occlusion. In the alternative, the medical device may not be bioerodable and can be retrieved after the drug dosage has been released.

The medical device of the present invention can take any form that can be implanted into the occlusion, such as a pellet or an open mesh type structure.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIGS. 1 and 2 are partial cross-sectional views illustrating potential problems associated with the treatment of chronic total occlusions.

FIG. 3 illustrates a guiding catheter assembly positioned within a patient's vasculature.

FIG. 4 is a cross-sectional view of the medical device of the present invention prior to implantation into the occlusion.

FIG. 5 is cross-sectional view of the medical device of the present invention during implantation into the occlusion.

FIG. 6 is a cross-section view of the medical device of the present invention after implantation into the occlusion.

FIG. 7 is a side view of an embodiment of the implant of the present invention.

FIG. 8 is a cross-sectional view of an embodiment of a coated implant of the present invention.

FIG. 9 is a perspective view of an embodiment of the implant of the present invention.

### DETAILED DESCRIPTION OF THE INVENTION

Specific embodiments of the present invention are now described with reference to the figures, where like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

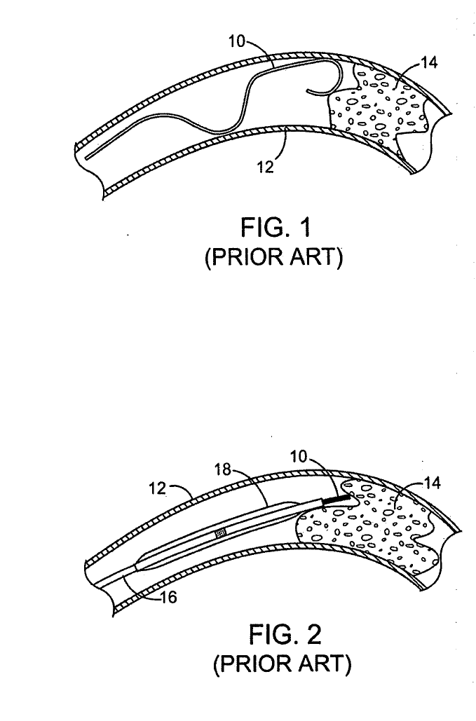

The present invention is directed to a drug-eluting device for treatment of chronic total occlusions. FIGS. 1 and 2 are cross-sectional views illustrating potential problems associated with the treatment of chronic total occlusions. Referring to FIG. 1, a standard or steerable guidewire 10 is advanced through a vessel 12 to the site of a chronic total occlusion 14. As depicted in FIG. 1, guide wire 10 may be unable to penetrate the proximal cap of occlusion 14 and may prolapse into vessel 12 when force is applied. Further, even if guidewire 10 can penetrate the proximal cap of occlusion 14, it may not be able to completely cross the occlusion.

FIG. 2 illustrates a prior art catheter 16 having a dilatation balloon 18 mounted thereon and the limitations of such when attempting to center a device such as guidewire 10 at the site of chronic total occlusion 14. As can be seen, guidewire 10 is not directed toward the center of occlusion 14, but in fact is undesirably directed toward the wall of vessel 12. Thus, difficulties may be encountered during attempts to traverse occlusion 14, and the risk of perforating vessel 12 may be increased.

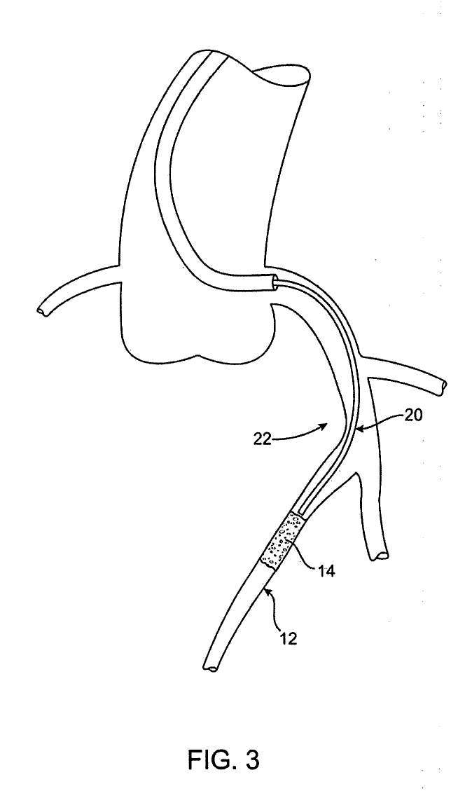

Referring to FIG. 3, a guiding catheter assembly 20 is shown positioned within a patient's vasculature. Typically, the guiding catheter assembly 20 is first inserted through an incision (not shown) and into a femoral artery of a patient. The assembly 20 is then advanced through the femoral artery into the patient's aorta and then into the ostium of the selected artery or vessel; for example, the left coronary artery 22. Guiding catheter assembly 20 is positioned by a physician, preferably with its distal end proximally adjacent to occlusion 14 in vessel 12.

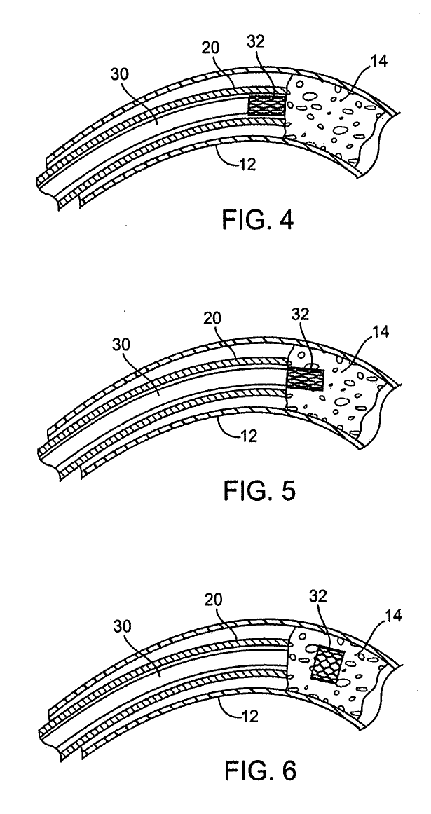

FIGS. 4-6 show cross-sections of an embodiment of the present invention at different stages of placement of a drug-eluting device into an occlusion. Referring to FIG. 4, guiding catheter 20 is advanced to a location proximal to occlusion 14. Advanced through catheter 20 is a pusher 30 and a drug-eluting implant 32. Pusher 30 may be a solid wire or a hypotube with an enclosed end in order to abut against an end of implant 32. Pusher 30 may also be made of a relatively high modulus, i.e. incompressible plastic material such as polyimide, polyester, polyamide, polyethylene block amide copolymer, or polyolefin, i.e. polypropylene, high density polyethylene (HDPE) or ultra-high molecular weight high density polyethylene (UHMW-HDPE). Elongate pusher 30 may vary in axial stiffness along its length such that a more distal portion may be sufficiently flexible to navigate through, or along with catheter 20, the typically more tortuous vasculature in the vicinity of the target occlusion. To accomplish varying stiffness with longitudinal incompressibility, pusher 30 may comprise varying transverse dimensions and/or a combination of various metals and/or plastic materials, as will be understood by those of skill in the art of medical guidewires.

As shown in FIG. 5, drug-eluting implant 32 is pushed into occlusion 14 by pusher 30. After drug-eluting implant 32 has been pushed into occlusion 14, implant 32 may expand so as to anchor itself within occlusion 14, as shown in FIG. 6. Implant 32 may expand due to absorption of fluid in the vessel. Alternatively, implant 32 may expand elastically, pseudo-elastically, or by thermal shape memory to a pre-formed shape. Pseudo-elastic properties or thermal shape memory properties may be achieved using an alloy such as nitinol. Implant 32 remains in occlusion 14 for a period of time to enable the drug to act upon the occlusion to soften or dissolve it. Thereafter, a conventional recanalization catheter procedure can be performed, such as balloon angioplasty and/or stenting. Due to the softening or dissolution of at least portions of the occlusion 14, a guidewire, and subsequently the treatment catheter, can pass through occlusion 14 for such a conventional recanalization procedure.

Implant 32 shown in FIGS. 4-6 is a lattice structure much like a stent. However, implant 32 is not required to have the same structure as a stent. For example, implant 32 does not require as much radial strength as a stent because it does not need to support the vascular wall.

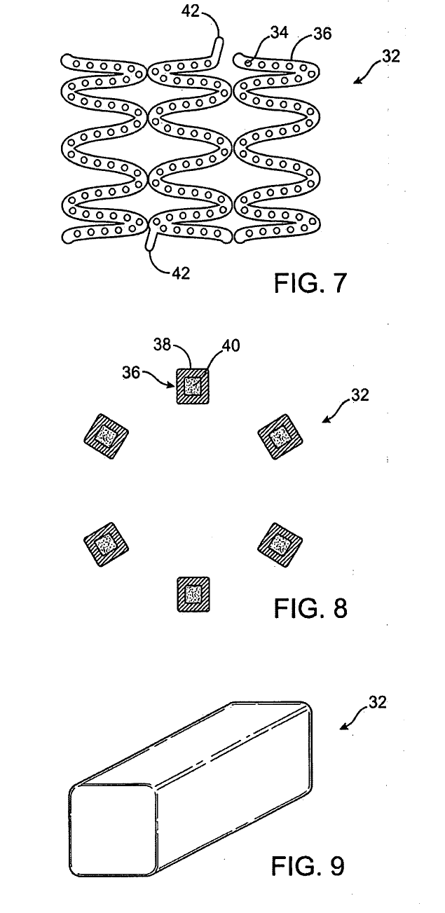

FIG. 7 shows an embodiment of implant 32 with stent-like structure including pores or openings 34 on struts 36 for storage of drug to be released into the occlusion. Openings 34 may penetrate the entire thickness of strut 36 or only a portion of the thickness of strut 36. Further, although implant 32 was described with respect to FIG. 6 as being self-expanding in order to be retained in occlusion 14, implant 32 does not need to expand. For example, the embodiment of FIG. 7 shows barbs 42 to anchor implant 32 within occlusion 14. Alternative structures or methods to retain implant 32 within occlusion 14 would be apparent to those skilled in the art.

FIG. 8 shows another embodiment of implant 32, wherein the drug to be released into occlusion 14 is stored in at least one coating layer 38 disposed around a base 40. Implant 32 can be made of any biocompatible material. Coating layer 38 may be made of a biodegradable polymer, for example, caprolactone, cellulose, collagen, albumin, casein, polysaccharides (PSAC), polylactide (PLA), poly-L-lactide (PLLA), polyglycol (PGA), poly-D,L-lactide-co-glycolide (PDLLA/PGA), polyhydroxybutyric acid (PHB), polyhydroxyvaleric acid (PHV), polyalkylcarbonate, polyorthoester, polyethylene terephthalate (PET), polymalic acid (PMLA), polyanhydrides, polyphosphazenes, polyamino acids and their copolymers as well as hyaluronic acid and derivatives thereof. Base 40 may comprise any of the biodegradable polymers listed above: regarding coating layer 38, or base 40 may include a non biodegradable polymer such as polyimide, polyester, polyamide, polyethylene block amide copolymer, or polyolefin. Such non biodegradable materials may need to be retrieved after implant 32 has been implanted for a pharmaceutically effective time.

Implant 32 can be made of metals including, but not limited to, gold, platinum, tantalum, iridium, tungsten, stainless steel, cobalt-chromium super alloy, titanium and alloys thereof. Such materials are not bioerodable and thus may need to be retrieved after implant 32 has been implanted for a pharmaceutically effective time. Alternatively, implant 32 can be made of a bioerodable metal, for example, magnesium and magnesium alloys such that implant 32 would not need to be retrieved. Instead, the implant 32 would dissolve in the vessel as it treats occlusion 14. Implant 32 can thus comprise various combinations of bioerodable, biodegradable or non-bioerodable or non-biodegradable materials to make coating layer 38 and base 40.

Although implant 32 has been shown as a stent-like structure, implant 32 can take on different forms, such as a sphere, a cylinder, a cone, a body having multiple prongs emanating from a center, an open geodesic structure such as a sphere or ovoid, or a solid polyhedral pellet shown in FIG. 9, as would be apparent to those skilled in the art.

The therapeutic formulation incorporated into implant 32 should be a drug that softens or dissolves the material of occlusion 14. The drug should be non-toxic or minimally toxic considering the small dosage delivered, and should not cause clotting of the blood. An example of the therapeutic formulation incorporated into implant 32 includes, but is not limited to, so-called “proteolytic enzyme-containing formulation” as described in U.S. Published Patent. Application Publication No. 2005/0053548. The proteolytic enzyme may be selected from: matrix metalloproteinases, serine elastases, trypsin, neutral protease, chymotrypsin, aspartase, cysteinase and clostripain. Matrix metalloproteinases (MMPs) is a group of zinc-containing enzymes that are responsible for degradation of extracellular matrix (ECM) components, including fibronectin, collagen, elastin, proteoglycans and laminin. These ECM components are important components of the occluding atherosclerotic plaque. MMPs play an important role in normal embryogenesis, inflammation, wound healing and tumour invasion. These enzymes are broadly classified into three general groups: collagenases, gelatinases and stromelysins. Collagenase is the initial mediator of the extracellular pathways of interstitial collagen degradation, with cleavage at a specific site in the collagen molecule, rendering it susceptible to other neutral proteases (e.g. gelatinases) in the extracellular space. In one embodiment, the proteolytic enzyme containing formulation includes a matrix metalloproteinase selected from: collagenase, type 1A collagenase, gelatinases, and stromelysins. In another embodiment, the proteolytic enzyme containing formulation includes collagenase, whether alone or in combination with other enzymes.

The therapeutic formulation incorporated into implant 32 can be a solubilizing agent, such as those discussed in U.S. Pat. No. 4,636,195 to Wolinsky, which is incorporated in its entirety by reference herein. For example, a therapeutic formulation including isotonic aqueous buffers containing phospholipids at a pH of from about 7.2 to 7.6 may be useful. Phospholipids are naturally available compounds that on hydrolysis yield fatty acids; phosphoric acid; an alcohol, usually glycerol; and a nitrogenous base such as choline or ethanolamine. They include lecithins, cephalins and sphingomyelins. Lecithins, particularly egg lecithin, are preferred because of their easy availability and efficiency. The efficiency of a formulation may be improved by the addition of bile acids such as cholic, deoxycholic, chenodeoxycholic, lithocholic, glycocholic and taurocholic acid. Addition of a collagenase, typically a mammalian collagenase, or one derived from bacteria may improve efficacy of the formulation. The collagenase cleaves the collagen that is the main supportive structure of the plaque, so that the plaque body then collapses. This result together with the solubilization of the fat and other components of the plaque serves to decrease markedly the total volume of the plaque. Other proteases such as papain, or chymotrypsin may also be employed together with the collagenase or as an alternative thereto. Other enzymes such as chondroitinase or hyaluronidase may also be employed alone or as one of the active components in the formulation liquid to assist in the removal of other plaque components.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1-17. (canceled)

18. A medical device comprising:
an implant configured to be implanted into a chronic total occlusion; and
a plaque softening or dissolving formulation contained in the implant, the implant being configured for administration of the formulation to the chronic total occlusion over a sustained time period sufficient to soften or dissolve at least a portion of the chronic total occlusion.

19. The medical device of claim 18, wherein the formulation is disposed in openings in the implant.

20. The medical device of claim 18, wherein the formulation is disposed in a coating disposed on the implant.

21. The medical device of claim 20, wherein the coating is a polymeric coating.

22. The medical device of claim 21, wherein the coating is selected from the group consisting of caprolactone, cellulose, collagen, albumin, casein, polysaccharides (PSAC), polylactide (PLA), poly-L-lactide (PLLA), polyglycol (PGA), poly-D,L-lactide-co-glycolide (PDLLA/PGA), polyhydroxybutyric acid (PHB), polyhydroxyvaleric acid (PHV), polyalkylcarbonate, polyorthoester, polyethyleneterephthalate (PET), polymalic acid (PML), polyanhydrides, polyphosphazenes, polyamino acids and their copolymers.

23. The medical device of claim 18, wherein the formulation is a proteolytic enzyme-containing formulation.

24. The medical device of claim 23, wherein the proteolytic enzyme is selected from the group consisting of matrix metalloproteinases, serine elastases, trypsin, neutral protease, chymotrypsin, aspartase, cysteinase and clostripain.

25. The medical device of claim 24, wherein the proteolytic enzyme-containing formulation comprises a matrix metalloproteinase selected from the group consisting of collagenase, type 1A collagenase, gelatinases, and stromelysins.

26. The medical device of claim 25, wherein the proteolytic enzyme-containing formulation comprises collagenase.

27. The medical device of claim 18, wherein the formulation includes isotonic aqueous buffers containing phospholipids.

28. The medical device of claim 27, wherein the phospholipids are selected from the group consisting of lecithins, cephalins and sphingomyelins.

29. The medical device of claim 18, further comprising a pusher for pushing the implant into the chronic total occlusion.

30. The medical device of claim 18, wherein the implant is expandable.

31. The medical device of claim 18, wherein the implant includes a barb for retaining the implant within the chronic total occlusion.

32. The medical device of claim 18, wherein the implant comprises a material selected from the group consisting of gold, platinum, tantalum, iridium, tungsten, stainless steel, cobalt-chromium super alloy, nickel, titanium, and alloys thereof.

33. The medical device of claim 18, wherein the implant comprises a bioerodable material.
